(19) United States
(12) Patent Application Publication
(10) Pub. No.: US 2002/0142474 A1
(43) Pub. Date: Oct. 3, 2002

(54) Contaminant library method and array plate

(76) Inventors: John Robert LaGraff, Niskayuna, NY (US); Xiao-Dong Sun, Mountain View, CA (US); James Claude Carnahan, Niskayuna, NY (US)

Correspondence Address: GENERAL ELECTRIC COMPANY, CRD PATENT DOCKET ROOM 4A59, P O BOX 8, BUILDING K 1 SALAMONE, SCHENECTADY, NY, 12301, US

(21) Appl. No.: 09/781,085

(22) Filed: Feb. 12, 2001

(51) Int. Cl. G01N 33/20

(52) U.S. Cl. 436/73; 436/79; 436/72; 436/84; 436/75; 436/83; 422/102; 134/42

(57) ABSTRACT

A method includes providing a substrate and depositing components of a test contaminant library onto regions of the substrate to form at least two test contaminant members of the library. In another method, a chemical cleaning solution is selected by combinatorial high throughput screening. In the method, components of a test contaminant library are deposited onto regions of a substrate to form at least two test contaminant members of the library. The substrate is cleaned with a cleaning solution and cleanliness of the substrate evaluated to select a cleaning solution for at least one of the contaminant members. In a final embodiment, the invention is a combinatorial high throughput screening array plate, comprising (A) a substrate and (B) a test contaminant library deposited on the substrate.

### BACKGROUND OF THE INVENTION

The present invention relates to a method of preparing a combinatorial test contaminant library. Particularly, it relates to a method and system and array plate for a high throughput screening (HTS) method to identify a cleaning solution using the contaminant library.

A typical gas turbine engine includes a compressor, a combustor and a turbine. Gases flow axially through the engine. Compressed gases emerging from the compressor are mixed with fuel and burned in the combustor. Hot products of combustion emerge from the combustor at high pressure and enter the turbine where thrust is produced to propel the engine and to drive the turbine, which in turn drives the compressor.

The compressor and the turbine include alternating rows of rotating and stationary coated airfoils. High temperature combustion gases degrade the coatings through either hot corrosion or oxidation. Gases that circulate through the airfoils, particularly during operation on the ground, also include contaminants such as dirt that has been ingested by the engine. Accumulation of dirt can impede effective cooling and if melted, can infiltrate and destroy thermal barrier coatings (TBC's).

The dirt typically comprises mixtures of Ca, Mg, Al, Si, Ni and Fe carbonates and oxides such as multi-elemental spinels (AB₂O₄). Dirt accumulation can cause serious damage at high engine operating temperatures. In particular, a low melting point eutectic Ca₃Mg₄Al₂Si₉O₃₀, (CMAS) similar in composition to diopside, can form from silicate-containing dirts at engine temperatures near 1200° C. and can wet and infiltrate coatings leading to crack formation and part failure. Other contaminants can include iron and nickel oxides, sodium vanadates, sodium sulfates, sodium phosphates and the like.

Other turbine engine part contaminants include thermally grown oxides (TGO). High temperature engine operation can result in TGO on coatings, which can unintentionally protect an underlying metal coating during chemical stripping. For example, alumina scales which form on metallic MCrAITY coatings impede chemical attack during stripping, thus leading to incomplete coating removal or excessive base metal attack, both of which can lead to either additional rework or part destruction. In one common repair scheme, TGO are first chemically or physically removed from the MCrAIY surface in order to facilitate subsequent coating removal with a chemical system. Other TGO systems include Cr₂O₃ and CoₓCr_{y}O spinels, which form on cobalt-based superalloys such as FSX414. These TGO can impede subsequent weld and braze repair processes. Consequently, it is important to periodically clean dirt and TGO from engine parts such as airfoils.

There is a need to select cleaning solutions for this purpose. Copending U.S. application SN (RD-27995) teaches a method of selecting a chemical cleaning solution by combinatorial high throughput screening (CHTS). The method can use a metal test coupon coated with a contaminant composition to select a best cleaning solution from an array of candidate cleaning solutions. However, this process is used to select a best cleaning solution only for a single dirt composition. The dirt composition is prepared by coating individual metal coupons and the cleaning solutions are determined by a combinatorial process to select a best cleaning solution with respect to the coated coupon. There is a need to quickly and efficiently find a best cleaning solution for a variety of test contaminants or to determine conditions for best cleaning for a variety of test contaminants.

### BRIEF SUMMARY OF THE INVENTION

The invention meets this need by providing a method of preparing a combinatorial test contaminant library. The method comprises providing a substrate and depositing components of a test contaminant library onto regions of the substrate to form at least two test contaminant members of the library. In another embodiment, the invention is a method for selecting a chemical cleaning solution by combinatorial high throughput screening, comprising depositing components of a test contaminant library onto regions of a substrate to form at least two test contaminant members of the library, cleaning the substrate with a cleaning solution and evaluating cleanliness of the substrate to select a cleaning solution for at least one of the contaminant members. In a final embodiment, the invention is a combinatorial high throughput screening array plate, comprising (A) a substrate and (B) a test contaminant library deposited on the substrate.

### BRIEF DESCRIPTION OF THE DRAWINGS

FIG. 1 is a flow chart of a method for selecting a chemical cleaning solution;

FIG. 2 is a schematic representation of a binary mask group; and

FIG. 3 is a schematic representation of an array plate with a test contaminant library.

### DETAILED DESCRIPTION OF THE INVENTION

The invention relates to a method for making dirt including TGO libraries on a metal substrate such as a superalloy or Pt foil. The invention can be utilized to make a library that mimics various contaminant compositions found on engine-run parts. The library can be used to test for best cleaning solutions. Particularly, the library can be used in a CHTS method to select best chemical cleaning solutions for turbine engine parts.

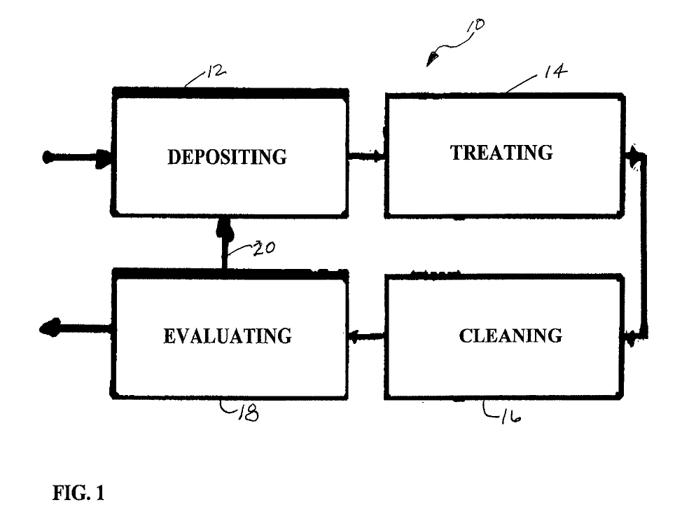

These and other features will become apparent from the drawings and following detailed discussion. FIG. 1 shows an overall method 10 for making dirt or TGO libraries on a substrate and screening of the substrate to select a cleaning solution. A combinatorial high throughput screening (CHTS) is preferred for selecting the cleaning solution. Typically, a CHTS is characterized by parallel reactions at a micro scale. Combinatorial chemistry techniques have been applied to search for new phosphors in thin film form or powder form. In the present invention, a combination of a thin-film deposition and masking strategy can be used to generate a thin film spatially addressable contaminant library, where each sample precursor in the library is formed from a multiple-layer. Following deposition of precursor layers, interdiffusion of the layers can be effected by a thermal annealing step and the resulting contaminant libraries cleaned and the cleaning solutions evaluated by determining cleaning extent of the libraries.

In FIG. 1, the method for selecting a chemical cleaning solution by CHTS includes steps of depositing 12 components of a contaminant library onto regions of a substrate to form at least two test contaminant members of the library, treating 14 the substrate with deposited contaminant by annealing or the like to simulate conditions of a used and dirtied engine part, cleaning 16 the substrate with a candidate cleaning solution and evaluating 18 cleanliness of the substrate to select a cleaning solution for at least one of the contaminant members.

In one aspect shown in FIG. 1, CHTS can be described as a method 10 comprising steps of (i) depositing 12 components of a test contaminant library onto regions of a substrate to form at least two test contaminant members of the library; (ii) cleaning 16 the substrate with a candidate cleaning solution under a selected reaction condition; and (iii) evaluating 18 a product of the cleaning step; and (B) reiterating 20 (A) wherein a successive solution or condition selected for a step (II) 16 is selected as a result of an evaluating step 16 (iii) of a preceding iteration of a step (A). A typical CHTS can utilize advanced automated, robotic, computerized and controlled loading, reacting and evaluating procedures.

The components of the test contaminant library can be deposited 12 by any suitable method. One method utilizes a multiple channel liquid dispensing system, wherein each of an array of liquid dispensers can be individually controlled and programmed to dispense a liquid material. In preferred embodiments, the liquid dispensers are each filled with a soluble metal precursor such as a nitrate, acetate or other aqueous soluble metal salt compound. An elemental metal, metal alloy or mixture thereof is carried in a soluble precursor.

The test contaminant library is deposited on a substrate that can be a metal usually used in engine parts. The substrate can be a button or coupon of airfoil material or other engine part material or it can be a simple metal or alloy plate. Examples of substrates include NiAl, PtAl, McrAlY and yttrium-stabilized zirconia, chromides, etc. coated superalloys. Examples of superalloys include Ni-based superalloys in both equiaxed and single crystal form, such as Rene N5, GTD111, etc. and Co-based alloys such as FSX414. Also, the contaminant library can be placed on Pt foils to minimize reactions between contaminant and substrate during high temperature intermixing of deposited oxides.

In a preferred embodiment, a thin film contaminant library on a substrate is produced using a multiple gun sputtering deposition system. The multiple gun sputtering deposition system contains a contaminant component placed in each gun cavity. An electrical discharge can be created at each source by applying radio frequency (RF) or direct current (DC) power in a range between about 10 Watts and about 1,000 Watts through the sputter gun, which heats the contaminant component to form a metal plasma vapor. The metal vapor from the sputter gun is deposited onto a counter-facing substrate. The rate of the material deposition is dependent on the level of power input. The amount of material deposited can be altered by changing the amount of time the sputter gun is powered.

By coupling thin film deposition from different sputter guns with different masking patterns from an array of deposition masks, a matrix library of thin film contaminants can be created. Due to the multiplicity of the number of guns and hence the metal contaminant components that can be used, the possible compositions and stoichiometry of contaminants which are deposited on the substrate are countless thus allowing for exploration of a vast experimental space. With multiple sputtering guns, any combination of metals can be deposited on a substrate to form the thin film contaminant library.

In various embodiments, the thin film contaminant library is built with an in-vacuum feed-in system. This enables the contaminant library to be made without breaking vacuum to change sources and masks for the next deposition, which keeps the metal contaminants in an atmospherically controlled environment. In particular, the in-vacuum feed-in system is filled with a gas, for example, argon, helium, nitrogen, hydrogen and mixtures thereof. The gas in the thin film contaminant library is referred to as “sputtering gas”. The in-vacuum feed-in system increases the speed of generation of libraries and also prevents the formation of metal oxides from elemental metals and alloys which are sensitive to oxygen.

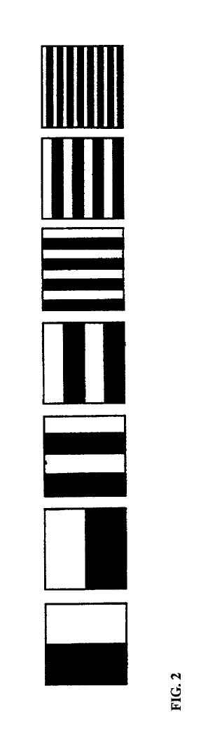

Preferably, a binary masking strategy is used in making the contaminant library. FIG. 2 is an example of a suitable binary mask group that can provide a binary masking strategy to prepare a substrate supported contaminant library. None of the binary masks are identical. In the process, approximately one half of a masking area is covered during each elemental deposition step. The masking strategy includes choice of mask form as well as masking procedure. Examples of mask forms include a shadow mask, a lithographic mask and a movable-shutter mask. The first two masks can be used for a broad search of contaminant systems while a shutter mask can be used for composition optimization in a discovered system of cleaning solutions. In the deposition process, a primary mask is applied to spatially divide the substrate. Then a sequence of secondary masks can be overlaid. Controlled quantities of various contaminant library components can be deposited through the secondary masks. The sequence and pattern of the secondary masks determine final composition of contaminant materials in the library.

FIG. 2 illustrates a suitable binary masking group. In binary masking, one half of a total primary masking area is covered on each elemental deposition step. The number of different contaminant library members compositions synthesized is 2ⁿ, where n is the number of operational steps. For example, 7 deposition steps represented by the 7 different masks of the group of FIG. 2, generate 128 (2⁷) different contaminant sample compositions on a substrate. Many possible combinations of the seven deposition entities can be created, from single elements, to binaries, ternaries, quaternaries, etc. An in-situ thickness monitor can be used to control the amount of material deposited from each sputtering gun.

Referring again to FIG. 1, treating step 14 can be a furnace annealing, furnace cycling (i.e., repeated heating and cooling) or a burner rig test, which involves cyclic exposure to hot combustion gas impingement. Generally the treating step 14 is carried out in an apparatus such as a furnace. In the furnace, the library is heated to a temperature in a range between about 200° C. and about 1100° C., and preferably, to a temperature in a range between about 600° C. and about 800° C. The hearing can be in a non-organic gas environment to substantially prevent oxidation of elemental metals or metal alloys. Examples of typical gas environments include argon, helium, nitrogen, hydrogen and mixtures thereof.

Referring again to FIG. 1, a solution can be used to clean 16 the library of contaminants to determine effectiveness of the solution for cleaning the wide variety of contaminants represented in the contaminant array. Extent and effectiveness of cleaning can then be evaluated 18 by a device that conducts an elemental analysis such as an energy dispersive spectroscopy apparatus, a cross-sectional metallography device or the like. Other examples of analyzers comprise a charge-coupled device (CCD) or analyzer camera that determines cleaning and effectiveness.

Another suitable piece of equipment to conduct the evaluating step 18 is an Eagle II Microfluorescence System (EDAX, Inc.), which uses X-rays to generate characteristic wavelength fluorescence that permits elemental identification to distinguish between coating and base metal. Another suitable analyzer 50 is based on “beat tint,” which involves oxidizing an entire coupon at several hundred degrees Celsius for an hour or two and observing a color change of the coating (or base metal). The color change identifies the amount of remaining coating or indicates whether the base metal has been completely exposed.

These and other features will become apparent from the following detailed discussion, which by way of example without limitation describes a preferred embodiment of the present invention.

### EXAMPLE

A combination of radio frequency (RF) sputtering and binary physical masking steps are used to generate a 128-member thin film dirt library, targeting various CMAS compositions. The sputtering targets (>99.9% purity) include CaCO₃, MgO, Al₂O₃, and SiO₂. The libraries are deposited on silicon, flat steel and Pt substrates. The amount of metals deposited are monitored in-situ with a quartz crystal thickness monitor. Subsequent analysis with a profilometer reveals that film thickness varies less than 5% over a two-inch diameter deposition area. The libraries are annealed in air from 800° C. to 1200° C. for 4 hours. The resulting coupon represents substantially all dirt deposits, which may be encountered in the field.

A compositional map of such library is shown in the following Table.

TABLE

| | | | |
|---|---|---|---|
| caCa0.5Mg0.5Mg1.5AlA10.5Si4.5 | caCa0.5Mg0.5AlA10.5Si4.5 | caMg0.5Mg1.5AlA10.5Si4.5 | caMg0.5AlA10.5Si4.5 |
| caCa0.5Mg0.5Mg1.5AlA10.5 | caCa0.5Mg0.5AlA10.5 | caMg0.5Mg1.5AlA10.5 | caMg0.5AlA10.5 |
| caCa0.5Mg0.5Mg1.5AlSi4.5 | Ca0.5Mg0.5AlSi4.5 | Mg0.5Mg1.5AlSi4.5 | caMg0.5AlSi4.5 |
| caCa0.5Mg0.5Mg1.5Al | Ca0.5Mg0.5Al | Mg0.5Mg1.5Al | caMg0.5Al |
| caCa0.5Mg0.5Mg1.5A10.5Si4.5 | Ca0.5Mg0.5A10.5Si4.5 | Mg0.5Mg1.5A10.5Si4.5 | caMg0.5A10.5Si4.5 |
| caCa0.5Mg0.5Mg1.5A10.5 | Ca0.5Mg0.5A10.5 | Mg0.5Mg1.5A10.5 | caMg0.5A10.5 |
| caCa0.5Mg0.5Mg1.5Si4.5 | caCa0.5Mg0.5Si4.5 | caMg0.5Mg1.5Si4.5 | caMg0.5Si4 5 |
| caCa0.5Mg0.5Mg1.5 | caCa0.5Mg0.5 | caMg0.5Mg1.5 | caMg0.5 |
| caCa0.5Mg1.5AlA10.5Si4.5 | caCa0.5AlA10.5Si4.5 | caMg1.5AlA10.5Si4.5 | caAlA10.5Si4.5 |
| caCa0.5Mg1.5AlA10.5 | caCa0.5AlA10.5 | caMg1.5AlA10.5 | caAlA10.5 |
| caCa0.5Mg1.5AlSi4.5 | caCa0.5AlSi4.5 | caMg1.5AlSi4.5 | caAlSi4.5 |
| caCa0.5Mg1.5Al | caCa0.5Al | caMg1.5Al | caAl |
| caCa0.5Mg1.5A10.5Si4.5 | caCa0.5A10.5S14.5 | caMg1.5A10.5Si4.5 | caA10.5Si4.5 |
| caCa0 5Mg1.5A10.5 | caCa0.5A10.5 | caMg1.5A10.5 | caA10.5 |
| caCa0.5Mg1.5Si4.5 | caCa0.5Si4.5 | caMg1.5Si4.5 | caSi4.5 |
| caCa0.5Mg1.5 | caCa0.5 | caMg1.5 | ca |
| Ca0.5Mg0.5Mg1.5AlA10.5Si4.5 | Ca0.5Mg0.5AlA10.SSi4.5 | Mg0.5Mg1.5AlA10.5S14.5 | Mg0.5AlA10.5Si4.5 |
| Ca0.5Mg0.SMg1.5AlA10.5 | Ca0.5Mg0.5AlA10.5 | Mg0.5Mg1.5AlA10.5 | Mg0.5AlA10.5 |
| Ca0.5Mg0.5Mg1.5AlSi4.5 | Ca0.5Mg0.5AlSi4.5 | Mg0.5Mg1.5AlSi4.5 | Mg0.5AlSi4.5 |
| Ca0.5Mg0.5Mg1.5Al | Ca0.5Mg0.5Al | Mg0.5Mg1.5Al | Mg0.5Al |
| Ca0.5Mg0.5Mg1.5A10.5Si4.5 | Ca0 5Mg0.5A10.5Si4.5 | Mg0.5Mg1.5A10.5Si4.5 | Mg0.5A10.5Si4.5 |
| Ca0.5Mg0.5Mg1.5A10.5 | Ca0.5Mg0.5A10.5 | Mg0.5Mg1.5A10.5 | Mg0.5A10.5 |
| Ca0.5Mg0.5Mg1.5Si4.5 | Ca0.5Mg0.5Si4 5 | Mg0.5Mg1.5Si4.5 | Mg0.5Si4.5 |
| Ca0.5Mg0.5Mg1.5 | Ca0.5Mg0.5 | Mg0.5Mg1.5 | Mg0.5 |
| Ca0.5Mg1.5AlA10.5Si4.5 | Ca0.5AlA10.5Si4.5 | Mg1.5AlA10.5Si4.5 | AlA10.5S14.5 |
| Ca0.5Mg1.5AlA10.5 | Ca0.5AlA10.5 | Mg1.5AlA10.5 | AlA10.5 |
| Ca0.5Mg1.5AlSi4.5 | Ca0.5AlSi4.5 | Mg1.5AlSi4.5 | AlSi4.5 |
| Ca0.5Mg1.5A1 | Ca0.5Al | Mg1.5Al | Al |
| Ca0.5Mg1.5A10.5Si4.5 | Ca0.5A10.5Si4 5 | Mg1.5A10.5Si4 5 | A10.5Si4.5 |
| Ca0.5Mg1.5A10.5 | Ca0.5A10.5 | Mg1.5A10.5 | A10.5 |
| Ca0.5Mg1.5Si4.5 | Ca0.5Si4.5 | Mg1.5S14 5 | Si4.5 |
| Ca0.5Mg1.5 | Ca0.5 | Mg1.5 | |

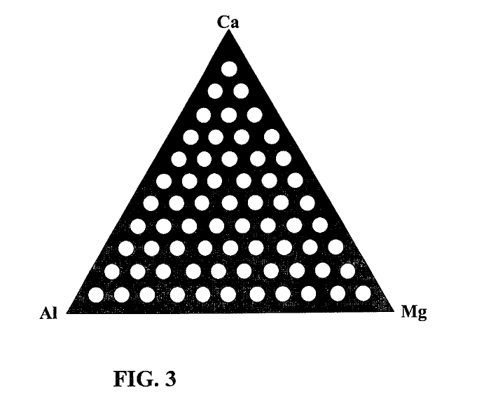

The method and array plate of the invention can be used to prepare contaminant libraries using sputtering and shadow masking with compositions ranging from single component oxides to binaries, ternaries, etc. FIG. 3 is a schematic representation of an array plate with a test contaminant library Ca, Mg and Al oxides of various compositions. The method and plate allow effective and rapid evaluation of test cleaning solutions. The method and plate result in (1) decreased development time for new chemical cleaning, (2) evaluation of a wide range of cleaning conditions and (3) rapid response to new cleaning and stripping problems.

While preferred embodiments of the invention have been described, the present invention is capable of variation and modification and therefore should not be limited to the precise details of the Examples. For example, the steps of depositing 12, treating 14, cleaning 16 and evaluating 18 can be reiterated 20 to provide complete test results on an experimental space. For example, the method can be conducted with three iterations using three different cleaning solutions to compare effectiveness of the solutions to clean identically dirtied engine parts. The invention includes changes and alterations that fall within the purview of the following claims.

What is claimed is:

1. A method of preparing a combinatorial test contaminant library, comprising providing a substrate and depositing components of a test contaminant library onto regions of said substrate to form at least two test contaminant members of said library.

2. The method of claim 1, further comprising utilizing said test contaminant library in a combinatorial high throughput screening (CHTS) method.

3. The method of claim 1, wherein said contaminant is a dirt.

4. The method of claim 1, wherein said contaminant is a CMAS.

5. The method of claim 1, wherein said contaminant is a silicate.

6. The method of claim 1, wherein said contaminant is selected from the group consisting of iron oxides, nickel oxides, sodium vanadates, sodium sulfates and sodium phosphates.

7. The method of claim 1, wherein said contaminant is a TGO.

8. The method of claim 1, wherein said contaminant is an alumina.

9. The method of claim 1, wherein said contaminant is a Cr₂O₃ spinel or CoₓCr_{y}O spinel.

10. A method for selecting a chemical cleaning solution by combinatorial high throughput screening, comprising:
depositing components of a test contaminant library onto regions of a substrate to form at least two test contaminant members of said library;
cleaning said substrate with a cleaning solution; and
evaluating cleanliness of said substrate to select a cleaning solution for at least one of said contaminant members.

11. The method of claim 10, comprising cleaning said substrate with a plurality of candidate cleaning solutions

12. The method of claim 10, comprising cleaning said substrate under a plurality of conditions

13. The method of claim 10, comprising depositing components of said test contaminant library in a manner to form masking sequenced compositions of said contaminant members on said substrate.

14. The method of claim 10, wherein said cleaning comprises applying said solutions to said test contaminant library in parallel.

15. The method of claim 10, comprising (A) steps of (i) depositing components of a test contaminant library onto regions of a substrate to form at least two test contaminant members of the library; (ii) effecting cleaning of said substrate with a candidate cleaning solution under a selected reaction condition; and (iii) evaluating a product of the cleaning step; and (B) reiterating (A) wherein a successive solution, condition or test contaminant library selected for a step (i) or step (II) is selected as a result of an evaluating step (iii) of a preceding iteration of (A).

16. The method of claim 10, wherein said contaminant is a dirt.

17. The method of claim 10, wherein said contaminant is a CMAS.

18. The method of claim 10, wherein said contaminant is a silicate.

19. The method of claim 10, wherein said contaminant is selected from the group consisting of iron oxides, nickel oxides, sodium vanadates, sodium sulfates and sodium phosphates.

20. The method of claim 10, wherein said contaminant is a TGO.

21. The method of claim 10, wherein said contaminant is an alumina.

22. The method of claim 10, wherein said contaminant is a Cr₂O₃ spinel or CoₓCr_{y}O spinel.

23. A combinatorial high throughput screening array plate, comprising; (A) a substrate and (B) a test contaminant library deposited on said substrate.

24. The array plate of claim 23, wherein said test contaminant library comprises a masking sequenced combination of components of at least two test contaminant members.

25. The array plate of claim 24, wherein said masking sequenced combination are produce by a binary masking sequence with respect to composition or thickness.

26. The array plate of claim 23, wherein said test contaminant library comprises uniform distribution of combinations of components of at least two test contaminant members.

27. The array plate of claim 23, wherein said test contaminant library comprises combinations of components of at least two test contaminant members deposited in quadrants.

28. The array plate of claim 23, wherein said contaminant is a dirt.

29. The array plate of claim 23, wherein said contaminant is a CMAS.

30. The array plate of claim 23, wherein said contaminant is a silicate.

31. The array plate of claim 23, wherein said contaminant is selected from the group consisting of iron oxides, nickel oxides, sodium vanadates, sodium sulfates and sodium phosphates.

32. The array plate of claim 23, wherein said contaminant is a TGO.

33. The array plate of claim 23, wherein said contaminant is an alumina.

34. The array plate of claim 23, wherein said contaminant is a Cr₂O₃ spinel or CoₓCr_{y}O spinel.
